(19) United States
(12) Patent Application Publication
(10) Pub. No.: US 2025/0325482 A1
(43) Pub. Date: Oct. 23, 2025

(54) Tenofovir Spray Drying Sachet Enema Powder Formulation for HIV Prevention

(71) Applicants: University of Pittsburgh - Of the Commonwealth System of Higher Education, Pittsburgh, PA (US); UPMC, Pittsburgh, PA (US); The Johns Hopkins University, Baltimore, MD (US)

(72) Inventors: Christina Bagia, Wexford, PA (US); Craig Hendrix, Ellicott City, MD (US); Lin Wang, Pittsburgh, PA (US); Lisa Cencia Rohan, Pittsburgh, PA (US)

(21) Appl. No.: 18/868,296

(22) PCT Filed: May 25, 2023
(86) PCT No.: PCT/US23/23521

§ 371 Date: Nov. 22, 2024

Related U.S. Application Data
(60) Provisional application No. 63/345,624, filed on May 25, 2022.

(51) Int. Cl. A61K 9/16; A61J 1/14; A61K 9/00; A61K 31/675; A61M 3/02

(52) U.S. Cl. CPC A61K 9/1611; A61J 1/1468; A61K 9/009; A61K 9/1682; A61K 31/675; A61M 3/0245; A61M 2202/064; A61M 2210/1067

(57) ABSTRACT

Provided herein are stable formulations containing active ingredients, such as antiviral compositions, or antiretroviral compositions in a powder form for reconstitution for intrarectal delivery to provide pre-exposure prophylaxis (PrEP) against viral infections. The antiviral composition may be tenofovir, for HIV PrEP.

### CROSS-REFERENCE TO RELATED APPLICATION

The present application claims priority to U.S. Provisional Patent Application No. 63/345,624, filed May 25, 2022, the content of which is incorporated herein by reference in its entirety.

### STATEMENT REGARDING FEDERALLY SPONSORED RESEARCH

This invention was made with government support under AI113127 awarded by the National Institutes of Health. The government has certain rights in the invention.

### BACKGROUND

Globally, the HIV epidemic continues to expand in men who have sex with men (MSM) and transgender women (TGW) for whom unprotected receptive anal intercourse is the primary HIV risk factor. Many individuals who engage in anal intercourse (AI) use cleansing douches regularly before and even after AI. Studies have shown that a rectal douche could be a good vehicle to deliver an HIV-protective drug in a behaviorally congruent manner.

Douching/enema use is a common procedure which men who have sex with men (MSM) perform before intercourse. However, it can also be a risk factor for HIV. Studies suggested that incorporating human immunodeficiency virus (HIV) prevention strategies into this behavior can provide an optimal means for drug administration. A locally-deliverable drug product for delivery of antiretroviral compound(s), such as tenofovir (TFV) is therefore desirable. To make the product more commercially viable and accessible to users without the need of pharmacy, and in terms of ease of use, storage stability, and distribution, a liquid enema product is not optimal. Improved dosage forms for delivery of tenofovir are therefore desirable.

### SUMMARY

A sachet powder product was developed utilizing a combination of the spray drying and powder compression to increase particle size and thus to improve powder flowability. The spray-dried powder was found to be stable, uniform and easily reconstituted in water. Therefore, provided herein is a drug product including a hermetically-sealed container; and a flowable powdered drug formulation, sealed within the container, the powdered drug formulation including particles ranging from 20 to 1000 in diameter and including smaller-sized particles of a dried antiretroviral compound ground with a salt or buffer.

Also provided herein is a method of preparing an antiviral pre-exposure prophylaxis drug product, including dissolving an antiretroviral compound in water to provide a solution, and adjusting the pH of the solution to a pH ranging from 6 to 8 or from 6.5 to 7.5, spray drying the solution under conditions for producing a powder comprising the antiretroviral compound, subjecting the antiretroviral compound to a dry granulation technique with a salt or buffer in amounts to produce, when dissolved in water to a volume of 125 mL, a prophylactically-effective concentration of the antiretroviral compound with an osmolality of 145±22 (±15%) milliosmoles per kilogram of water (mOsm/kg H2O), e.g., using a freezing point micro osmometer.

Further embodiments are set forth in the following numbered clauses:

Clause 1. A drug product comprising: a hermetically-sealed container; and a flowable powdered drug formulation, sealed within the container, the powdered drug formulation comprising particles ranging from 20 to 600 in diameter comprising smaller-sized particles of a dried antiretroviral compound ground with a salt or buffer.

Clause 2. The drug product of clause 1, wherein the ionic salt or buffer is an alkali metal chloride.

Clause 3. The drug product of clause 1 or clause 2, wherein the ionic salt or buffer is sodium chloride.

Clause 4. The drug product of any of clauses 1-3, wherein the antiretroviral compound is a nucleoside reverse transcriptase inhibitor, a nonnucleoside reverse transcriptase inhibitor, a protease inhibitor, a fusion inhibitor, an entry inhibitor, or an integrase strand transfer inhibitor.

Clause 5. The drug product of any of clauses 1-4, wherein the antiretroviral compound is tenofovir, or a pharmaceutically acceptable salt thereof.

Clause 6. The drug product of clause 5, wherein the tenofovir is in the form of tenofovir disoproxil, tenofovir alafenamide, and/or tenofovir exalidex.

Clause 7. The drug product of clause 5, wherein the tenofovir is 9-[9(R)-2-(phosphonomethoxy)propyl]adenine (PMPA).

Clause 8. The drug product of any of clauses 5-7, comprising an amount of tenofovir and an amount of sodium chloride to yield, when reconstituted in water, a hypotonic solution of from 145 mOsm/kg to about 290 mOsm/kg comprising from 0.1 mg/ml to 20 mg/ml, 1.8 mg/ml to 10 mg/ml, 2 mg/ml to 10 mg/ml, or 5.28 mg/ml, of PMPA or a therapeutic or molar equivalent amount of a different form of tenofovir, such as tenofovir disoproxil, tenofovir alafenamide, and/or tenofovir exalidex

Clause 9. The drug product of any of clauses 5-8, comprising a multiple of 660 mg±66 of PMPA.

Clause 10. The drug product of any of clauses 1-9, wherein the container comprises a cylindrical profile.

Clause 11. The drug product of any of clauses 1-10, wherein the container comprises a polyester, such as a biaxially-oriented polyethylene terephthalate, that is optionally metallized.

Clause 12. The drug product of any of clauses 1-11, wherein the container is a stick pack comprising a tear notch at an end.

Clause 13. The drug product of any of clauses 1-12, wherein the container is a sachet comprising a tear notch at an end.

Clause 14. The drug product of any of clauses 1-13, wherein the flowable powdered drug formulation comprises 0.2-1.6 TFV:NaCl.

Clause 15. The drug product of any of clauses 1-14, comprising at least 500 mg of tenofovir.

Clause 16. The drug product of any of clauses 1-15, providing, when reconstituted in water to a concentration of 5.28 mg/mL of PMPA, pharmacokinetic levels meeting or exceeding ICgo value for human immunodeficiency virus over 24 hours.

Clause 17. The drug product of any of clauses 1-16, providing, when reconstituted in water to a concentration of 5.28 mg/mL of tenofovir, an osmolality of 145±22 (±15%) milliosmoles per kilogram of water (mOsm/kg 1120), e.g., using a freezing point micro osmometer.

Clause 18. A kit comprising, the drug product of any of clauses 1-17 and an enema bottle or enema bag and/or a container comprising sterile water.

Clause 19. A method of preparing an antiviral pre-exposure prophylaxis drug product, comprising: dissolving an antiretroviral compound in water to provide a solution, and adjusting the pH of the solution to a pH ranging from 6 to 8 or from 6.5 to 7.5; spray drying the solution under conditions for producing a powder comprising the antiretroviral compound; grinding the antiretroviral compound with a salt or buffer in amounts to produce, when dissolved in water to a volume of 125 mL, a prophylactically-effective concentration of the antiretroviral compound with an osmolality of 145±22 (±15%) milliosmoles per kilogram of water (mOsm/kg H2O), e.g., using a freezing point micro osmometer.

Clause 20. The method of clause 19, wherein the spray drying is performed under the following conditions: inlet temperature set to about 200° C., pump flow set to about 4 m/min, aspirator set to about 35 m3/h, and N2 flow set to about 601 L/h.

### BRIEF DESCRIPTION OF THE DRAWINGS

FIG. 1 shows exemplary stick packs (top) and sachets (bottom) according to non-limiting embodiments described herein;

FIG. 2 shows an exemplary stick pack according to non-limiting embodiments described herein;

FIG. 3 shows exemplary stick packs according to non-limiting embodiments described herein; and

FIG. 4 shows exemplary stick packs according to non-limiting embodiments described herein.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise” or “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of HIV means administration to a patient by any suitable dosage regimen, procedure and/or administration route of a composition, device, or structure with the object of achieving a desirable clinical/medical end-point, including but not limited to, for HIV, reducing or preventing infection and/or transmission of HIV, e.g., for “pre-exposure prophylaxis (PrEP). An amount of any reagent or therapeutic agent, administered by any suitable route, effective to treat a patient is an amount capable of preventing, reducing, and/or eliminating HIV infection and/or transmission. The therapeutically-effective amount of each therapeutic may range from 1 μg per dose to 10 g per dose, including any amount there between, such as, without limitation, 1 ng, 1 g, 1 mg, 10 mg, 100 mg, or 1 g per dose. The therapeutic agent may be administered by any effective route, and, for example, as a single dose or bolus, at regular or irregular intervals, in amounts and intervals as dictated by any clinical parameter of a patient, or continuously.

Active ingredients, such as an antiretroviral composition, may be compounded, formulated, or otherwise manufactured into a suitable composition for use, such as a pharmaceutical dosage form, a rectal dosage form, or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. Additional non-limiting examples of useful excipients include disaccharides or sugar polyols (e.g., lactose, glucose, sorbitol, and maltitol).

Useful dosage forms include: intrarectal, intravenous, intramuscular, intraocular, or intraperitoneal solutions, oral tablets or liquids, topical ointments or creams and transdermal devices (e.g., patches). In the context of the present disclosure, suitable dosage forms may include single-dose, or multiple-dose sachets, vials, or other containers for dry-powder drug products. The dry powder drug product may be reconstituted in any useful container for ultimate delivery in liquid form as an enema or douche. Suitable containers for delivery include, without limitation, an enema bag, an enema bottle, an enema bottle having an extended tip, medical syringes or droppers, containing a composition comprising an active ingredient useful for PrEP. Additional dosage forms may include a rectal dosage form configured in a liquid dosage form, a solid dosage form, or a semi-solid dosage form.

As indicated above, the drug product described herein is provided as a powder that is reconstituted by an end-user in water to produce a hypoosmotic composition comprising a prophylactically-effective amount of an antiretroviral drug, such as tenofovir, for example and without limitation ranging from 0.1 mg/ml to 20 mg/ml, 1.8 mg/ml to 10 mg/ml, 2 mg/ml to 10 mg/ml, or 5.28 mg/ml, of tenofovir, tenofovir alafenamide or tenofovir disoproxil, tenofovir exalidex (CMX-157), or equivalent amounts of another pharmaceutically-effective tenofovir salt, or more generally an equivalent amount of a different antiretroviral drug, for example and without limitation, a nucleoside reverse transcriptase inhibitor, a nonnucleoside reverse transcriptase inhibitor, a protease inhibitor, a fusion inhibitor, an entry inhibitor, or an integrase strand transfer inhibitor.

The powder dosage form may be reconstituted in an appropriate amount of water, which can be sterile or non-sterile prior to use. Water includes tap water, spring water, well water, purified or filtered water, distilled water, double-distilled water, deionized water (DI), reverse osmosis-(RO) purified water, RO/DI water, and any other form of water that can safely be used in an enema product and contains insufficient salt to raise the ionic strength of the solution after addition of the powder drug product to physiologically isotonic or hypertonic levels. The powder dosage form may be provided in a kit with a suitable container, such as an enema bottle or enema bag, and optionally water.

Therapeutic compositions may be sterile, and should be stable under the conditions of manufacture and storage. For example, sterile powders can be prepared by incorporating the active agent in an appropriate solvent with one, or a combination of ingredients, followed by filter-sterilization. The liquid may be spray-dried, vacuum dried, lyophilized, or otherwise dried, under aseptic conditions, and packaging, such as sachets or other containers, may be sterilized prior to filling with the powder described herein under aseptic conditions. Once filled, the sachet or other container may be further sterilized, e.g., in a manner that does not, or does not substantially deteriorate the powdered drug product within the sachet or other container.

A “therapeutically effective amount” refers to an amount of a drug product or active agent effective, at dosages and for periods of time necessary, to achieve the desired therapeutic result. An “amount effective” for treatment of a condition or for prophylaxis is an amount of an active agent or dosage form, such as a single dose or multiple doses, effective to achieve a determinable end-point. The “amount effective” is preferably safe—at least to the extent the benefits of treatment outweigh the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide an optimum desired response (e.g., a therapeutic or prophylactic response), and/or to maintain desirable therapeutic and/or prophylactic levels of an active agent. In non-limiting embodiments, the present devices, kits, and methods provide a desirable level of active agent for up to 144 hours in vivo. For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, every three days, every four days etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals. In the context of the powdered product described herein, the powder may be reconstituted in a suitable delivery device, such as an enema bag or enema bottle, and may be delivered, e.g., intrarectally, as a single dose, or as multiple doses over a given time period, such as 2, 3, 4, or more doses administered, for example and without limitation, every 10, 15, 20, 30, 45, 60, 90, or 120 minutes, every 2 through 12 hours daily, or every other day, or any increment therebetween, or, in the case of three or more doses, over different time intervals, such as, for example, administering the second dose 30 minutes after the first dose, and administering subsequent doses once a day thereafter.

Provided herein are stable pharmaceutical compositions (e.g., drug products) in the form of a dry powder, optionally included in a stick pack or sachet, and further optionally included in a kit, that may be reconstituted as a hypotonic, isotonic, and/or iso-osmolar liquid formulation, of various therapeutic compositions that may be advantageously delivered using a rectal delivery device (e.g., an enema bag or enema bottle). The formulation comprises an antiretroviral compound, e.g. tenofovir, and a salt or buffer to produce a hypotonic solution comprising an effective amount of the antiretroviral compound.

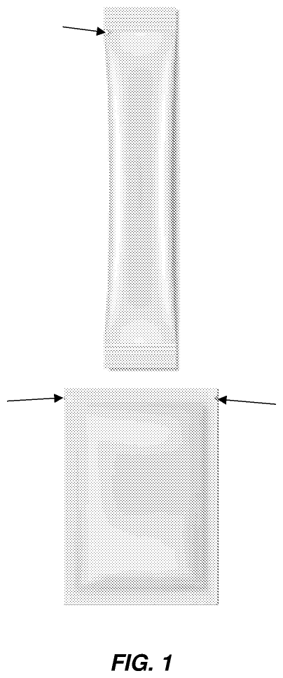

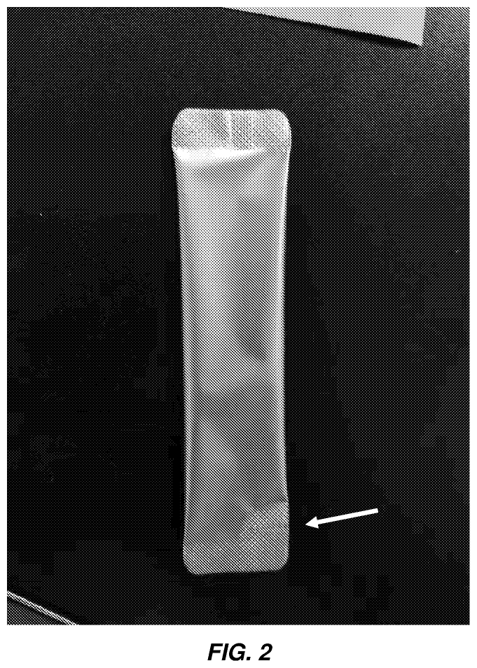

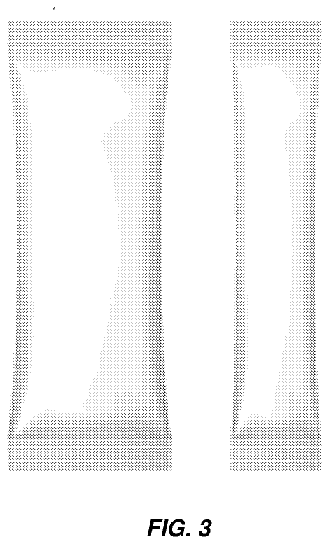

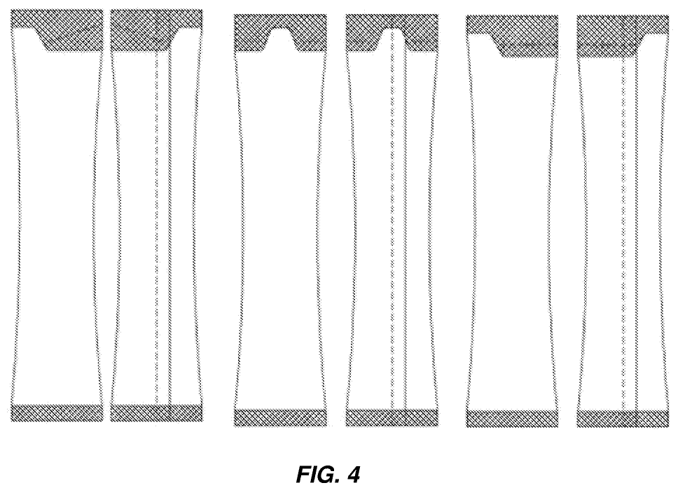

FIG. 1 shows exemplary sachets according to non-limiting embodiments described herein. FIGS. 1-4 show exemplary stick packs according to non-limiting embodiments described herein. Stick packs and sachets are common packaging products in many industries, including the food and pharmaceutical industries. They typically comprise, e.g., are formed from, a polymeric membrane, such as Mylar or a Mylar foil laminate. Stick pack pouches can comprise an elongated tube-shaped, e.g., cylindrical pouch with a fold-over side and an optional tear notch, which can be formed from tubular materials that are sealed at both ends. The tube may have a continuous side seam. A stick pack typically has a length at least three or at least four times the width or diameter of the tube. Stick packs may include a tear notch, arranged at any suitable location to allow for a user to easily open the pack. Sachets are typically flat or bulging pouches sealed at three or four sides, often including a tear notch, which may be arranged at any suitable location to allow for a user to easily open the sachet. The tear notch may be adjacent to a spout portion of the sachet extending into the seal and configured to provide an opening narrower than a dimension of the sachet. Non-limiting embodiments of suitable packaging, including non-limiting locations of tear notches, are shown in the images of FIGS. 1-4 (arrows and dashed lines identifying non-limiting embodiments of locations and arrangements for tear notches).

Tonicity is a measure of the effective osmotic pressure gradient, for example, the water potential of two solutions separated by a semipermeable cell membrane. Tonicity depends on the relative concentration of selectively membrane permeable solutes across a cell membrane which determine the direction and extent of osmotic flux. It is commonly used when describing the swelling versus shrinking response of cells immersed in an external solution. A hypotonic solution has a lower concentration of solutes than another solution. In biology, a solution outside of a cell is called hypotonic if it has a lower concentration of solutes relative to the cytosol. Due to osmotic pressure, water diffuses into the cell, and the cell often appears turgid, or bloated. For cells without a cell wall such as animal cells, if the gradient is large enough, the uptake of excess water can produce enough pressure to induce cytolysis, or rupturing of the cell. A hypertonic solution has a greater concentration of solutes than another solution. In biology, the tonicity of a solution usually refers to its solute concentration relative to that of another solution on the opposite side of a cell membrane. A solution outside of a cell is called hypertonic if it has a greater concentration of solutes than the cytosol inside the cell. When a cell is immersed in a hypertonic solution, osmotic pressure tends to force water to flow out of the cell in order to balance the concentrations of the solutes on either side of the cell membrane. The cytosol is conversely categorized as hypotonic, opposite of the outer solution.

In some embodiments, the formulation is hypotonic and has an osmolality from about 20 mOsm/kg to about 280 mOsm/kg, from about 50 mOsm/kg to about 250 mOsm/kg, from about 75 mOsm/kg to about 225 mOsm/kg, from about 100 mOsm/kg to about 200 mOsm/kg, about 110 mOsm/kg to about 180 mOsm/kg, about 123 mOsm/kg to about 167 mOsm/kg, about 137 mOsm/kg, about 139 mOsm/kg, or about 145 mOsm/kg. In non-limiting embodiments, the formulation is isotonic, having an osmolality of up to 300±42 mOsm/kg.

As used herein, a rectal delivery device is a device used to deliver the formulations or compositions described herein to the rectum. Traditional rectal delivery devices have been used for localized treatments including delivery of laxatives, treatment of hemorrhoids, and for delivery of antipyretics. Rectal delivery devices include, but are not limited to, an enema bag or an enema bottle, e.g., having an extended tip.

In some embodiments, the therapeutic compositions/active ingredients of the formulation useful in the rectal delivery device described herein can include, but not limited to, antiretroviral compositions (e.g., nucleoside reverse transcriptase inhibitors, nonnucleoside reverse transcriptase inhibitors, protease inhibitors, fusion inhibitors, entry inhibitors, and integrase strand transfer inhibitors, such as, for example and without limitation, efavirenz, emtricitabine, rilpivirine, atazanavir sulfate, darunavir ethanolate, elvitegravir, lamivudine, zidovudine, abacavir, zalcitabine, dideoxycytidine, azidothymidine, didanosine, dideoxyinosine, stavudine, rilpivirine, etravirine, delvaridine, nevirapine, amprenavir, tipranavir, inidinavir, saquinavir, lopinavir, ritonavir, fosamprenavir, ritonavir, darunavir, atazanavir, nelfinavir, enfuvirtide, raltegravir, dolutegravir, elvitegravir, maraviroc, DS003, tenofovir (TFV), tenofovir exalidex (CMX-157), TFV alafenamide, TFV disoproxil fumarate, and dapivirine), antiviral compositions (e.g., 4′-Ethynyl-2-fluoro-2′-deoxyadenosine (EFdA), nucleoside analogs, such as: acyclovir (2-amino-9-(2-hydroxyethoxymethyl)-3H-purin-6-one), penciclovir (2-amino-9-[4-hydroxy-3-(hydroxymethyl)butyl]-3H-purin-6-one), foscarnet (phosphonoformic acid), cidofovir ([(2S)-1-(4-amino-2-oxopyrimidin-1-yl)-3-hydroxypropan-2-yl]oxymethylphosphonic acid), adefovir (2-(6-aminopurin-9-yl)ethoxymethylphosphonic acid), and pharmaceutically-acceptable ester prodrugs thereof, such as valaciclovir (valine aciclovir ester, 2-[(2-amino-6-oxo-3H-purin-9-yl)methoxy]ethyl (2S)-2-amino-3-methylbutanoate) or famciclovir ([2-(acetyloxymethyl)-4-(2-aminopurin-9-yl)butyl]acetate)), antibiotic/antiprotozoal compositions (e.g., GRFT, CSIC, metronidazole), antifungal compositions (e.g., clotrimazole), hormones or hormonal compositions (e.g., levonorgestrel, etonogrestrel, desogrestrel, dienogest), hormonal receptor modulators (e.g., ulipristal acetate), and bases or salts of the foregoing. In addition, other compounds such as RANTES derivatives and retrocyclin (e.g., RC-101) can be included. Compositions that affect metabolism of another composition, such as antiretroviral compositions, such as cobicistat (sold under the trade name Tyboost®) can also be included. For example, a composition can include atazanavir and cobicistat (sold under the trade name Evotaz®).

In some embodiments, the therapeutic composition comprises tenofovir or a pharmaceutically acceptable salt thereof. In some embodiments, the therapeutic composition is tenofovir, tenofovir alafenamide, or tenofovir disoproxil. In some embodiments, the active ingredient of the therapeutic composition, e.g., tenofovir, tenofovir alafenamide, CMX-157 (base or salt), or tenofovir disoproxil, is present in an amount from 0.1 mg/ml to 20 mg/ml, 1.8 mg/ml to 10 mg/ml, 2 mg/ml to 10 mg/ml, or 5.28 mg/ml. In non-limiting embodiments, the active ingredient is present in an amount exceeding 1.76 mg/ml. In non-limiting embodiments, the active ingredient is present in an amount of at least about 5.28 mg/ml, optionally at least about 10.28 mg/ml.

The formulation may be a composition comprising an antiretroviral compound and a salt or buffer co-mingled with the antiretroviral compound in a dry state. For example, the a liquid-dissolved antiretroviral compound may be spray-dried or lyophilized to produce particles of 20-1000 microns, optionally 20-600 microns, for example less than 50μ (microns), less than 40μ, less than 30μ, less than 25μ, or less than 20μ in diameter or particles or less than 50μ (microns), less than 40μ, less than 30μ, less than 25μ, or less than 20μ average diameter, as determined by any effective means. In non-limiting embodiments, the particles, following spray drying, are non-flowing, and have a particle size of less than 20μ. The dried particles may be subsequently ground or processed with a dry granulation technique together with a salt, buffer, or other excipient (e.g., mannitol or microcrystalline cellulose) to produce larger, flowable particles of, e.g., greater than 50μ, greater than 60μ, greater than 75μ, greater than 90μ, or greater than 100μ in diameter, or greater than 50μ, greater than 60μ, greater than 75μ, greater than 90μ, or greater than 100μ in average diameter, as determined by any effective means. Any useful statistical method may be used to determine average particle size or particle size distribution for a population of particles. Particle size may be measured by, for example and without limitation, sieve analysis, microscopy, light scattering (e.g., dynamic light scattering, laser diffraction, among many other technologies and methods. Particle size analyzers are broadly-available commercially.

In one embodiment, a composition comprising tenofovir is provided. A solution of tenofovir is dissolved in water, and is neutralized to pH 7.0±1. In other non-limiting embodiments, a solution of TFV disoproxil or TFV alafenamide is dissolved in water to provide a solution with a final pH of 5.2-6, or CMX-157 (potassium form) was dissolved in water to provide solution with a final pH of 7.0±1

The tenofovir solution may be neutralized with NaOH. Other bases may be used to neutralize the tenofovir, such as KOH or Mg(OH)₂. A sodium phosphate buffer also may be used to neutralize the tenofovir, followed, optionally, by grinding or dry granulation with sodium phosphate (rather than NaCl, as described below). The neutralized solution is lyophilized or spray-dried, as is common in the pharmaceutical industry. The particles may be comminuted if lyophilized to produce a more uniform particle size distribution. Tenofovir+NaOH particles are quite hygroscopic and do not flow well. As such, after drying the particles are maintained in a low-humidity environment of air, an inert gas such as Ar, or nitrogen gas, having a low relative humidity, for example a relative humidity of less than 35%, 30%, 25%, or 20%, such as ranging from 20% to 25%, including any integer or increment therebetween. The dried tenofovir is then ground with a salt, such as NaCl, monosodium phosphate, disodium phosphate, potassium monobasic phosphate, potassium dibasic phosphate, potassium chloride, magnesium chloride, or a mixture of any of the preceding, such as a mixture of monosodium phosphate and disodium phosphate, to produce flowable particles that comprise intermingled tenofovir and salt. In non-limiting embodiments, another excipient, such as a sugar, may be added to improve flowability. The particles may be then packaged, e.g., in a sachet or other container. In non-limiting embodiments, tenofovir, at a concentration of 110 mg/mL (e.g., 11% w/w), may be prepared with NaOH, and spray drying may be performed to provide a powder, where spray drying may be performed with the inlet set at about 200° C., pump flow set at about 4 mL/min, aspirator set at about 35 m³/h, and N₂ flow set at about 601 L/h. When dried and combined with sodium chloride crystals, a powder with suitable followability may be achieved.

The formulation may be, for example, a stable pharmaceutical composition in the form of a hypotonic formulation for the delivery of tenofovir (including tenofovir, analog or derivatives thereof, or different pharmaceutical salt forms). Tenofovir may be provided as 9-[9(R)-2-(phosphonomethoxy)propyl]adenine (PMPA, referred to herein as tenofovir), tenofovir disoproxil, as tenofovir alafenamide, or as tenofovir exalidex. The salt forms, for example fumarate salt forms, of either compound can be included in compositions as described herein. For ease of reference, references herein will be made to “tenofovir” or TFV, with the understanding that the term can refer to PMPA, tenofovir disoproxil or tenofovir alafenamide, as well as salts thereof.

In non-limiting embodiments, the formulation consists of, that is, the formulation includes only, TFV, NaCl, NaOH, and, optionally if needed to adjust pH, HCl and/or NaOH, where the dose of TFV, when reconstituted, is 5.28 mg/mL.

### Example 1

For the formulation development of a tenofovir (TFV) enema sachet powder, several excipients were tested (Table 1).

TABLE 1

| Excipients | Role |
|---|---|
| Sodium chloride (NaCl) | Osmolality |
| Sodium hydroxide (NaOH) | pH modifier |
| Sodium hydrogen carbonate (NaHCO₃) | pH modifier |
| Ethylenediaminetetraacetic acid (EDTA) | Chelating agent |
| Methocel E5 Premium LV Hydroxypropyl | Flowing property |
| Methylcellulose (Methocel E5) | improvement |
| Methocel E15 Industrial LV Hydroxypropyl | Flowing property |
| Methylcellulose (Methocel E15) | improvement |
| Sodium dibasic phosphate (Na₂HPO₄) | pH modifier and osmolality |
| Sodium monobasic phosphate (NaH₂PO₄) | pH modifier and osmolality |

Sodium chloride (NaCl) was utilized to control the osmolality of the enema solution (after the powder is dissolved in water). Sodium hydroxide (NaOH) and sodium hydrogen carbonate (NaHCO₃) were used to establish the pH of the enema solution at approximately pH 7. Ethylenediaminetetraacetic acid (EDTA) was tested as chelating agent for the discoloration issues that appeared during the development. Finally, Methocel E5 and E15 low viscosity were evaluated in order to improve the flowability of the spray-dried powders. Finally, as alternative pH modifier and osmolality regulator sodium dibasic phosphate (Na₂HPO₄) and sodium monobasic phosphate (NaH2PO4) were utilized.

Three different manufacturing processes were investigated for the development of tenofovir sachet powder form: a) Dry Mixing; b) Lyophilization; and c) Spray Drying. Additionally, different packaging materials were evaluated with different processes powders.

### Dry Mixing

With the dry mixing procedure, different powder combinations were tested short-term for powder appearance, drug content, pH, and osmolality in accelerated conditions (65° C.) using different packaging materials (transparent glass vials, amber color glass vials and foil pouches (polyester/foil laminates RFE-042)). Initially, two doses were prepared for each test formulation: low dose/Iso-osmolar (LD/I_{SO}) and high dose/Iso-osomolar (HD/I_{SO}). Table 2 shows the different formulations of dry powders that were prepared and tested.

TABLE 2

| Formula- | | TFV | NaCl | NaOH | NaHCO₃ | EDTA |
|---|---|---|---|---|---|---|
| tion# | Dose | (mg) | (g) | (mg) | (g) | (g) |
| 1 | LD/Iso* | 220 | 1.078 | 45 | — | — |
| 2 | HD/Iso* | 660 | 0.996 | 135 | — | — |
| 3 | LD/Iso* | 220 | — | 45 | — | — |
| 4 | HD/Iso* | 660 | — | 135 | — | — |
| 5 | LD/Iso* | — | 1.078 | 45 | — | — |
| 6 | HD/Iso* | — | 0.996 | 135 | — | — |
| 7 | HD/Iso* | 660 | — | — | — | — |
| 8 | LD/Iso** | 220 | 0.8287 | — | 0.42 | — |
| 9 | HD/Iso** | 660 | — | — | 1.26 | — |
| 10 | HD/Iso** | 660 | 0.996 | 135 | — | 0.0625 |
| 11 | HD/Iso** | 660 | 0.996 | 135 | — | 0.125 |
| 12 | HD/Iso** | 660 | 0.996 | 135 | — | 0.625 |

The clinical formulation (TFV/NaCl/NaOH) that was prepared for LD/Iso and HD/Iso levels (formulations #1 and 2 in Table 2), showed powder discoloration for both dry powders on day 7 (pink patches throughout the powder), and afterwards the powders became beige in color, for both glass vials (transparent and amber color). On the other hand, in the foil pouch, it was noted that the same powder turned yellow after 7 days. Controls were prepared to understand better the source of discoloration: i) TFV+NaOH, ii) NaCl+NaOH and iii) TFV alone (formulations #3, 4, 5, 6 and 7 described in Table 2). The discoloration of the powder was only noted on the TFV+NaOH combination. Ethylenediaminetetraacetic acid (EDTA) was used in different amounts (0.05%, 0.1% and 0.5%) to potentially eliminate the discoloration (formulations #10, 11 and 12 in Table 2). It was observed that in the glass vial containers, some discoloration of the powder (off white) was noted but only for the lowest amount of EDTA (0.05%) but for all three EDTA levels, the powders turned into a hard mass (like a rock). On the other hand, in the foil pouches the powders with 0.05% and 0.1% EDTA turned yellow after 14 days at 65° C. After all these observations, it was decided do not pursue EDTA as a potential excipient for the TFV sachet formulation. Further, more formulations were evaluated and NaHCO₃ was tested as pH modifier (as an alternative of NaOH). It was noted that the foil pouches were found bloated after a few days (formulations #8 and 9 in Table 2) as well as minor pH increase of the liquid enema (pH=7.9). Overall, the dry mixing formulations were found unstable (discoloration), not uniform and not easily reconstituted. For these reasons, the dry mixing process was not adequate for the TFV sachet powder formulation.

### Lyophilization

Various powder combinations were tested for appearance, drug content, pH and osmolality in accelerated conditions (65° C.) utilizing different packaging materials: transparent glass vials and foil pouches (polyester/foil laminates RFE-042). Initially, two doses were prepared for each test formulation: low dose/Iso-osmolar (LD/I_{SO}) and high dose/Iso-osomolar (HD/I_{SO}). Table 3 shows the different combinations of lyophilized powders that were tested at the pre-formulation stage.

TABLE 3

| Formulation | | TFV | NaCl | NaOH | NaHCO₃ |
|---|---|---|---|---|---|
| # | Dose | (mg) | (g) | (mg) | (g) |
| 17 | LD/Iso | 220 | 1.078 | 45 | — |
| 18 | HD/Iso | 660 | 0.996 | 135 | — |
| 19 | LD/Iso | 220 | 0.8287 | — | 0.42 |
| 20 | HD/Iso | 660 | — | — | 1.26 |
| 21 | HD/Hypo | 660 | 0.4125 | 135 | — |

Four formulations (LD/Iso and HD/I_{SO}) were evaluated: two clinical (TFV/NaCl/NaOH) and two TFV/NaCl/NaHCO₃ formulations (formulations #17, 18, 19 and 20 in Table 3). The lyophilized powders showed no discoloration (compared to dry mixing). With NaHCO₃, no sachets were found bloated (compared to respective dry mixing powders). However, the pH and the drug content of the reconstituted powder enemas with NaHCO₃ were slightly elevated. Overall, the lyophilized formulations were found to be stable, uniform and could be easily reconstituted.

Optimization of the process was performed for scale-up production to be more efficient, less time consuming, and to produce a free flowing uniform powder. Briefly, different amounts of water (125, 80, 30 and 10 mL) were investigated to prepare the initial liquid formulation before the lyophilization. For this purpose, 10 mL of water was chosen. Moreover, V-blender (type of blender with a V shape) with stainless steel spheres was utilized to break the produced lyophilized cakes to form free flowing powders. As a final step for the powder to be uniform in size, sieves with specific mesh sizes and sieve shaker were utilized in order to collect 20-600 μm in size.

With the updated lyophilization process, a high dose/hypo-osmolar (HD/Hypo) TFV/NaOH/NaCl enema sachet powder formulation was prepared (Product C, formulation #21 in Table 3). The sachet powder formulation was packaged in polyester/foil laminates (RFE-042) from Amcor and monitored for stability per ICH guidelines for 24 months under three storage conditions, 25° C./60% RH, 30° C./65% RH and 40° C./75% RH. The enema powder was tested for drug content, pH, osmolality, water content, and visual observations. The lyophilized powder was found stable with all tested parameters remained within set specifications.

Additional X-ray diffraction (XRD) measurements were performed at selected time points (3 and 12 months). TFV was shown to maintain its amorphous state for the clinical formulation regardless the storage temperature. Additional studies were conducted to study hygroscopicity of the TFV enema powder, crystallinity using scanning electron microscopy (SEM), XRD, flowability (according to USP <1174>) and fineness of powders based on the USP <811> to better characterize the product properties. Results indicated that the powder was highly hygroscopic, therefore environmental humidity during production needs to be controlled (20-25% RH), and the sachet product would need to be used within 24 hours once the package is teared open. Through the SEM and XRD studies, TFV in the clinical formulation was found to be in amorphous state.

According to the fineness study, it was found that the majority of the powder in the clinical formulation was “very fine” (60%), the rest of the powder was distributed between “coarse” (8%), “moderate fine” (20%) and “fine” (13%). Additionally, the flowability of the lyophilized powder was considered as “passable”.

### Spray Dry

HD/Hypo TFV/NaOH/NaCl clinical formulation (Product C) was investigated for the development of the spray drying powder (formulation #22 from Table 4) and it was conducted in the following stages.

TABLE 4

| Formula- | | TFV | NaCl | NaOH | Methocel | Methocel |
|---|---|---|---|---|---|---|
| tion # | Dose | (mg) | (g) | (mg) | E5 (g) | E15 (g) |
| 22 | HD/Hypo | 660 | 0.4125 | 135 | — | — |
| 23 | HD/Hypo | 660 | 0.4125 | 135 | 1.8 | — |
| 24 | HD/Hypo | 660 | 0.4125 | 135 | | 1.8 |
| 25 | HD/Hypo | 660 | 0.4125 | 135 | 0.45 | |
| 26 | HD/Hypo | 660 | 0.4125 | 135 | 0.225 | |
| 27 | HD/Hypo | 660 | 0.4125 | 135 | 0.1 | |

Firstly, the primary goal was to obtain powder enema with high yield and low water content. For this purpose, various parameters of the spray dryer including inlet temperature, pump flow rate, aspirator, nitrogen (N₂) gas flow rate and water volume of the formulation were evaluated (Run #1-12 from Table 5).

TABLE 5
TFV/NaOH/NaCl

| | | | | | | water | | water |
|---|---|---|---|---|---|---|---|---|
| | Tᵢₙₗₑₜ | Tₒᵤₜₗₑₜ | | | N₂ | volume | Nozzle/ | content (%)* |
| Run # | (° C.) | (° C.) | aspirator % | pump % | (mmHg) | (mL) | Comments | Yield % ** |
| 1 | 160 | 96 | 90 | 20 | 50 | 10 | default nozzle | 5.35 |
| | | | | | | | 0.7 mm | |
| 2 | 160 | 73 | 90 | 40 | 50 | 10 | default nozzle | 7.02 |
| | | | | | | | 0.7 mm | |
| 3 | 180 | 100 | 90 | 20 | 50 | 10 | default nozzle | 4.40 |
| | | | | | | | 0.7 mm | |
| 4 | 180 | 114 | 90 | 40 | 50 | 10 | default nozzle | 4.98 |
| | | | | | | | 0.7 mm | |
| 5 | 180 | 109 | 90 | 10 | 50 | 10 | default nozzle | 3.92 |
| | | | | | | | 0.7 mm | |
| 6 | 180 | 109 | 90 | 10 | 50 | 6 | default nozzle | 3.28 |
| | | | | | | | 0.7 mm | |
| 7 | 180 | 109 | 90 | 10 | 50 | 30 | default nozzle | 2.94 |
| | | | | | | | 0.7 mm | |
| 8 | 180 | 112 | 90 | 10 | 50 | 80 | default nozzle | 2.67 |
| | | | | | | | 0.7 mm | |
| 10 | 200 | 113 | 90 | 20 | 50 | 10 | default nozzle | 2.88 |
| | | | | | | | 0.7 mm | |
| 11 | 200 | 88 | 90 | 40 | 50 | 10 | default nozzle | 3.76 |
| | | | | | | | 0.7 mm | |
| 12 | 200 | 115 | 90 | 15 | 50 | 10 | default nozzle | 2.89 |
| | | | | | | | 0.7 mm | |
| 13 | 180 | 117 | 90 | 10 | 60 | 10 | Ultrasonic | 3.32 |
| | | | | | | | nozzle 9 W | |
| | | | | | | | power level | |
| 14 | 180 | 127 | 90 | 2 | 60 | 10 | Ultrasonic | 3.48 |
| | | | | | | | nozzle 6.5 W | |
| | | | | | | | power level | |
| 15 | 180 | 117 | 90 | 10 | 60 | 10 | Ultrasonic | 3.50 |
| | | | | | | | nozzle 6.0 W | |
| | | | | | | | power level | |
| 16 | 150 | 110 | 90 | 3 | 55 | 10 | Ultrasonic | 3.48 |
| | | | | | | | nozzle 6.5 W | Yield: |
| | | | | | | | power level | 45.5% |
| 17 | 150 | 98 | 80 | 10 | 50 | 10 | Ultrasonic | 5.73 |
| | | | | | | | nozzle: 7.0 W | Yield: |
| | | | | | | | power level | 26.2% |
| 18 | 165 | 105 | 80 | 10 | 50 | 10 | Ultrasonic | 4.06 |
| | | | | | | | nozzle 7.0 W | Yield: |
| | | | | | | | power level | 27.1% |
| 19 | 175 | 110 | 80 | 8 | 50 | 10 | Ultrasonic | 3.65 |
| | | | | | | | nozzle 6.5 W | Yield: |
| | | | | | | | power level | 31.1% |
| 20 | 175 | 120 | 100 | 10 | 50 | 10 | Ultrasonic | 3.25 |
| | | | | | | | nozzle 5.5 W | Yield: |
| | | | | | | | power level | 23.5% |
| 21 | 190 | 129 | 100 | 10 | 50 | 10 | Ultrasonic | 2.53 |
| | | | | | | | nozzle 6.5 W | Yield: |
| | | | | | | | power level | 28.5% |
| 22 | 180 | 121 | 90 | 10 | 50 | 10 | Ultrasonic | 2.80 |
| | | | | | | | nozzle 90% | Yield: |
| | | | | | | | load mode | 25.3% |
| 23 | 180 | 125 | 90 | 5 | 50 | 10 | Ultrasonic | 2.45 |
| | | | | | | | nozzle 91% | Yield: |
| | | | | | | | load mode | 36.8% |
| 24 | 150 | 106 | 95 | 2 | 55 | 10 | Ultrasonic | 4.20 |
| | | | | | | | nozzle 8.00 W | Yield: |
| | | | | | | | power level | 42.7% |
| 25 | 140 | 98 | 80 | 2 | 50 | 10 | Ultrasonic | 4.60 |
| | | | | | | | nozzle 5.80 W | Yield: |
| | | | | | | | power level | 43.3% |
| 26 | 160 | 108 | 80 | 2 | 50 | 10 | Ultrasonic | 3.49 |
| | | | | | | | nozzle 5.80 W | Yield: |
| | | | | | | | power level | 46.5% |
| 27 | 200 | 120 | 90 | 15 | 50 | 10 | default nozzle | 3.47 |
| | | | | | | | 0.7 mm | |
| | | | | | | | Methocel E5 | |
| | | | | | | | 12% w/w | |
| 28 | 200 | 117 | 90 | 15 | 50 | 10 | default nozzle | 3.30 |
| | | | | | | | 0.7 mm | |
| | | | | | | | Methocel E5 | |
| | | | | | | | 12% w/w | |
| 29 | 200 | 117 | 90 | 15 | 50 | 10 | default nozzle | — |
| | | | | | | | 0.7 mm | |
| | | | | | | | Methocel E5 | |
| | | | | | | | 6% w/w | |
| 30 | 200 | 116 | 90 | 15 | 50 | 10 | default nozzle | — |
| | | | | | | | 0.7 mm | |
| | | | | | | | Methocel E5 | |
| | | | | | | | 6% w/w | |
| 31 | 200 | 114 | 90 | 15 | 50 | 10 | default nozzle | — |
| | | | | | | | 0.7 mm | |
| | | | | | | | Methocel E5 | |
| | | | | | | | 1.6% w/w | |
| 32 | 200 | 117 | 90 | 15 | 50 | 6 | default nozzle | — |
| | | | | | | | 0.7 mm | |
| | | | | | | | TFV/NaOH | |

*Water content was tested on the spray-dried powder.
** Yield (%) of the spray-dried powder is only reported for the ultrasonic nozzle since it was the lowest. For the default nozzle the Yield achieved was around 70-80%.

The optimal settings and volume were chosen based on the product outcome (Run #12 from Table 5). The resulting spray-dried product was overall acceptable physicochemically, nevertheless it was found to be sticky to the packaging pouches due to the fineness (<20 μm particle size).

Secondly, flowability improvement was investigated. For this stage, the impact of particle size of the dry powder on flowability was tested. The product made using an ultrasonic nozzle yielded larger particle size and the flowability of the powder was improved as compared to the prototype (Run #13-26 from Table 5). However, this approach was not pursued further considering the practicality of scale up, for the following reasons: 1) process was not continuous, e.g. 3 times per day needed to be stopped to change the N₂ gas tank; 2) the production yield was low (around 40-45%), and 3) typical CMOs do not have this capability.

Thirdly, utilization of the original nozzle was investigated, and different strategies for particle size and flowability improvement were explored. Approaches included modification of the formulation composition and combination of processes. For example, addition of other excipients such as methocel E5 and E15 (formulations #23-27 from Table 4) significantly increased the flowability and particle size (Run #27-31 from Table 5). Yet, the reconstituted enema from this powder, even though the osmolality did not increase, produced a foamy solution, which at this stage was not acceptable based on the current specifications. Lastly, a combination approach for formulation #22 from Table 4 (Product C) was evaluated, which involved producing a spray dried TFV with sodium hydroxide powder first (Run #32 from Table 5) and then combining that powder with sodium chloride crystals with the aid of grinding techniques. The resulting powder had an improved flowability and the rest of the physicochemical parameters were within specifications (powder appearance, water content, crystallinity, solution appearance, drug content, pH and osmolality). Flowability of this powder was evaluated (based on USP 1174) and the flow was considered as “very poor”. The optimized spray-dried powder was utilized for packaging evaluation.

The optimized spray dried sachet powder formulation was packaged in home-made stick packs modified from silver Mylar Foil Bags (PAKVF4) (from IMPAK) and placed to monitor the stability per ICH guidelines for 12 months under three storage conditions, 25° C./60% RH, 30° C./65% RH and 40° C./75% RH. The enema powder is tested for drug content, pH, osmolality, water content and visual observations. Initial stability points were tested and the produced powder is stable under at least the follow conditions: 25° C./60% RH and 40° C./75% RH for at least 6 months. More stability studies are planned for the spray dried powder.

Several packaging materials, size and shapes were evaluated for this project (Table 6).

TABLE 6

| Pouch material | Company | Size/Type | Comments |
|---|---|---|---|
| Silver Mylar Foil | IMPAK | 1.4″ × 4.0″/3-way seal | Not suitable |
| Bags (PAKVF4) | Corporation | | (flat pouch) |
| White Mylar Foil | IMPAK | 1.4″ × 6.0″/3-way seal | Not suitable |
| Bags | Corporation | | (too long + |
| | | | flat) |
| Black Mylar Foil | IMPAK | 1.75″ × 2.5″/4-way | Not suitable |
| Mini Pouch | Corporation | seal | (small + flat + |
| | | | corner and |
| | | | edges) |
| Green Mylar Foil | IMPAK | 4.25″ × 6″/4-way seal | Not suitable |
| Pouch | Corporation | | (big + flat + |
| | | | corner and |
| | | | edges) |
| 48 ga Met Pet/2.5 | Macopkg | 1.5″ × 4.0″/4-way seal | Not suitable |
| Mil LLDPE | | | (flat + corner |
| | | | and edges) |
| Cad Pak P | Macopkg | 1.5″ × 4.0″/4-way seal | Not suitable |
| 35440-D | Amcor | 1.5″ × 4.5″/3-way seal | Not suitable |
| RFA-044 | Amcor | 1.4″ × 4.0″/3-way seal | Not suitable |
| | | | (flat pouch) |
| Silver Mylar Foil | IMPAK | 1.0″ × 3.6″/stick | Suitable |
| Bags (PAKVF4) | Corporation | pouch | |
| RFA-044 | Amcor | 1.0″ × 3.6″/stick | Suitable |
| | | pouch | |

Briefly, different sizes and types of Mylar bags were evaluated from IMPAK and it was noted that the 3-way sealing was a better option for the product with a 4.00″ length. 3-way sealed Mylar bags with sizes 1.4″×4.0″ and 1.4″×6.0″ were readily available to purchase. From Macopkg, Met Pet pouches were ordered and purchased but the packages were 4-way sealed which was characterized as not suitable for the product. From Amcor, two different packages types were tested (35440-D and RFA-044). RFA-044 was identified as more suitable.

Both lyophilized and spray-dried powders were tested comparing Mylar bags with Amcor RFA-044. Powder recoveries were evaluated for 3-way flat sealed and in-house modified stick pouch. Results showed that, for the lyophilized product, the powder recovery from both companies stick pouches were equally good (95%) whereas for the flat original packaging the RFA-044 was a little better compared to Mylar bags (92% and 82% respectively). Similarly, the optimized spray-dried powder with improved flowability, stick pouches gave better powder recoveries (90%) compared to 3-way flat pouches (75-80%). Due to packaging availability, Mylar bags 1.4″×4.0″ were further advanced and were additionally in-house modified as stick pouches for both lyophilized and spray-dried Product C.

### Example 2

Tenofovir (TFV) solution was prepared by adding 145.2 g of TFV to a beaker containing 1155 g of water. 165 mL of 18% NaOH was then added while stirring. Mixing continued for 40 minutes until all TFV was dissolved. pH was adjusted, with 1N HCl or 1N NaOH to achieve a pH of 7.0±0.5. The solution was then spray dried with a Buchi B290 spray dryer, with the following settings:
Inlet Temp: 200° C.;
Pump: 15%
Aspirator: 90%
N₂: 50 mmHg

When the spray dryer was equilibrated, the TFV solution was fed into the pump, and spray drying continued until there was no remaining solution. The humidity of the spray drying room was maintained at 22-23%.

The powder that was produced was weighed, indicating that a yield of 77.7% was obtained.

4.125 g of NaCl was placed into a mortar and 7.45 g of the powder was added. The mixture was further processed using grinding or a dry granulation technique, and was thereafter transferred to a closed container.

### Example 3

### Preparation of Powder Enema Samples

Liquid formulation 110 mg/mL (pre-spray dryer): To 50 μL of TFV solution (110 mg/mL), 950 μL of water was added, to provide a total volume of 1000 μL of enema solution (resulting concentration of 5.5 mg/mL). This was prepared in triplicate. Then, to 20 μL of the 5.5 mg/mL enema solution, 980 μL of water was added, to provide a total volume of 1000 μL. The final concentration was 110 μL/mL. This was used for HPLC analysis.

Powder formulation post-spray drying (0.66 g of TFV in 0.795 g total dry powder): Approximately 60 mg of spray dried powder was placed in a 20 mL scintillation vial and, and 10 mL of water was added. The vial was capped and the solution was mixed well for around 10-15 times. Five difference samples were prepared, and the amount of TFV/NOH needed to combine with 0.4125 g of NaCl was calculated. The theoretical dry powder was 0.795 g/dose. The necessary amount was typically about 0.745 g (0.71-0.78 g).

Powder formulation was prepared by combining spray-dried TFV/NaOH with NaCl (0.66 g of TFV in 1.1575 g total dry powder) using a grinding or dry granulation technique. Approximately 90 mg of powder was added to a 20 mL scintillation vial, and 10 mL of water was added. The vial was capped and the solution was mixed well for around 10-15 times. Ten different samples were prepared. The amount needed to fill 1 sachet was calculated, taking into consideration the powder yield from the sachet (how much powder can be recovered from the sachet).

### Final Product (Sachets):

Drug content: A sachet was opened and emptied into a 250 mL glass bottle. The sachet was not rinsed, rather only mechanical steps (e.g., tapping) were taken to empty the sachet. 125 mL of MilliQ water was added to the 250 mL bottle, which was capped and mixed for around 20 times. A clear colorless solution (5.28 mg/mL TFV) was formed. The process was then repeated two more times.

To assess uniformity of content, 10 sachets were prepared as described above and analyzed.

For UPLC analysis, to 20 μL of the above TFV enema solution 980 μL of MilliQ water was added, to provide a total volume of 1000 μL. The final theoretical concentration of TFV was 105.6 μg/mL.

### Example 4

Tenofovir (TFV) solution iss prepared by adding 12,012.0 g of TFV to a 150 L tank containing 95,550.0 g of water. 16,107.0 g of 18% NaOH is then added while stirring. Mixing continues for 1 hour or until TFV is fully dissolved. pH is measured and, if necessary, pH is adjusted, with 1N HCl or 1N NaOH to achieve a pH of 7.0±0.5. The solution is then spray dried with a MS150 spray dryer, utilizing the following settings:
Inlet Temp: 185-195° C.
Outlet Temp: 100-110° C.
Pump (spray rate): 50-60 g/min
Fan Speed: 100%
Chamber pressure: 30 mmH₂O
Oxygen level: ≤5%
Cold Condenser Temp: 5° C.

When the spray dryer is equilibrated, the TFV solution is fed into the pump, and spray drying continues until there is no remaining solution. The humidity of the spray drying room is maintained at 30-40%. The TFV spray dried (TFV SDD) powder that is produced is weighed, indicating that a yield of 50-60% was obtained.

A secondary drying step is added for the TFV SDD powder utilizing a vacuum oven set at 65° C. for no less than 72 hours.

3,753.75 g of NaCl powder, which is milled prior to use, is placed into a 15 L bin blender with 6,779.5 g of the TFV SDD powder. The powders are blended for 1 hour and the uniformity of both TFV and NaCl are tested by UPLC and osmolality testing.

After uniformity of materials is established, a dry granulation is performed by using roller compaction process. Briefly, the powder mixture (TFV SDD with NaCl) is fed to the roller compactor and with adequate parameters, ribbons of powder are formed. The ribbons are then passed through 20 mesh sieve and the produced powder is flowable and the particle size is 20-650 um. The final flowable powder is fully characterized for powder appearance, drug content, uniformity of content, pH, osmolality, particle size, flowability and XRD. The packaging material to be used is a stick pouch with a combination of PET+AL+PE layers with 80 micron thickness. Stability per ICH guidelines for 24 months under three storage conditions, 25° C./60% RH, 30° C./65% RH and 40° C./75% RH are monitored.

Having described this invention, it will be understood to those of ordinary skill in the art that the same can be performed within a wide and equivalent range of conditions, formulations and other parameters without affecting the scope of the invention or any embodiment thereof.

1. A drug product comprising:
a hermetically-sealed container; and
a flowable powdered drug formulation, sealed within the container, the powdered drug formulation comprising particles ranging from 20μ to 600μ in diameter comprising smaller-sized particles of a dried antiretroviral compound ground with a salt or buffer.

2. The drug product of claim 1, wherein the ionic salt or buffer is an alkali metal chloride.

3. The drug product of claim 1, wherein the ionic salt or buffer is sodium chloride.

4. The drug product of claim 1, wherein the antiretroviral compound is a nucleoside reverse transcriptase inhibitor, a nonnucleoside reverse transcriptase inhibitor, a protease inhibitor, a fusion inhibitor, an entry inhibitor, or an integrase strand transfer inhibitor.

5. The drug product of claim 1, wherein the antiretroviral compound is tenofovir, or a pharmaceutically acceptable salt thereof.

6. The drug product of claim 5, wherein the tenofovir is in the form of tenofovir disoproxil, tenofovir alafenamide, and/or tenofovir exalidex.

7. The drug product of claim 5, wherein the tenofovir is 9-[9(R)-2-(phosphonomethoxy)propyl]adenine (PMPA).

8. The drug product of claim 5, comprising an amount of tenofovir and an amount of sodium chloride to yield, when reconstituted in water, a hypotonic solution of from 145 mOsm/kg to about 290 mOsm/kg comprising from 0.1 mg/ml to 20 mg/ml, 1.8 mg/ml to 10 mg/ml, 2 mg/ml to 10 mg/ml, or 5.28 mg/ml, of PMPA or a therapeutic or molar equivalent amount of a different form of tenofovir.

9. The drug product of claim 8, wherein the different form of tenofovir is tenofovir disoproxil, tenofovir alafenamide, and/or tenofovir exalidex.

10-11. (canceled)

12. The drug product of claim 1, wherein the container comprises a polyester.

13. The drug product of claim 12, wherein the polyester is a biaxially-oriented polyethylene terephthalate.

14. The drug product of claim 13, wherein the biaxially-oriented polyethylene terephthalate is metallized.

15. The drug product of claim 1, wherein the container is a stick pack comprising a tear notch at an end.

16. The drug product of claim 1, wherein the container is a sachet comprising a tear notch at an end.

17. The drug product of claim 1, wherein the flowable powdered drug formulation comprises 0.2-1.6 TFV:NaCl.

18. (canceled)

19. The drug product of claim 1, wherein, when reconstituted in water to a concentration of 5.28 mg/mL of PMPA, the drug product provides pharmacokinetic levels meeting or exceeding IC₉₀ value for human immunodeficiency virus over 24 hours.

20. The drug product of claim 1, wherein, when reconstituted in water to a concentration of 5.28 mg/mL of tenofovir, the drug product exhibits an osmolality of 145±22 (±15%) milliosmoles per kilogram of water (mOsm/kg H₂O).

21. A kit comprising, the drug product of claim 1 and an enema bottle or enema bag and/or a container comprising sterile water.

22. (canceled)

23. A method of preparing an antiviral pre-exposure prophylaxis drug product, comprising:
dissolving an antiretroviral compound in water to provide a solution, and adjusting the pH of the solution to a pH ranging from 6 to 8 or from 6.5 to 7.5;
spray drying the solution under conditions for producing a powder comprising the antiretroviral compound;
grinding the antiretroviral compound with a salt or buffer in amounts to produce, when dissolved in water to a volume of 125 mL, a prophylactically-effective concentration of the antiretroviral compound with an osmolality of 145±22 (±15%) milliosmoles per kilogram of water (mOsm/kg H₂O).

24. The method of claim 23, wherein the spray drying is performed under the following conditions: inlet temperature set to about 200° C., pump flow set to about 4 mL/min, aspirator set to about 35 m³/h, and N₂ flow set to about 601 L/h.
